(19) United States
(12) Patent Application Publication
(10) Pub. No.: US 2012/0215124 A1
(43) Pub. Date: Aug. 23, 2012

(54) NON-INVASIVE ARTERIAL BLOOD GAS DETERMINATION

(76) Inventors: Joseph Fisher, Thornhill (CA); James Duffin, Toronto (CA); Jorn Fierstra, Toronto (CA)

(21) Appl. No.: 13/367,225

(22) Filed: Feb. 6, 2012

Related U.S. Application Data
(60) Provisional application No. 61/439,731, filed on Feb. 4, 2011.

(51) Int. Cl. A61B 5/08; A61M 16/20; A61M 16/00

(52) U.S. Cl. 600/532

(57) ABSTRACT

A breathing circuit for use in conjunction with a ventilator serving a mechanically-ventilated patient includes an expiratory gas airflow pathway; an inspiratory gas airflow pathway; and a gas mixing mechanism operable to mix inspiratory gas and expiratory gas in an amount sufficient to equilibrate the patient's PETCO₂ and arterial PCO₂ such that the patient's PETCO₂ is a clinically reliable approximation of the patient's PaCO₂.

### FIELD OF THE INVENTION

The present invention is concerned with methods and devices for evaluating partial pressures of blood gases in ventilated patients, and in ventilated and spontaneously breathing patients with pulmonary disease or a systemic condition which (or the treatment of which) affects the distribution of blood flow in the lung or the distribution of ventilation or both.

### BACKGROUND OF THE INVENTION

During critical care, monitoring acid-base balance and the adequacy of ventilation, requires repeated invasive measurements of the partial pressure of CO₂ in arterial blood (PaCO₂) especially during weaning from mechanical ventilatory support. These place critically ill patients at risk for such associated complications as anemia¹, infection², arterial catheter blockage, and vascular endothelial injury and thrombosis. These risks are especially high in pediatric patients in whom the circulatory blood volumes, arteries and arterial catheters are smaller than in adults. In addition, drawing, transporting, and analyzing the samples consume considerable health care resources^{1;3}.

By contrast, measuring the partial pressure of CO₂ in end-tidal gas (PETCO₂) is a non-invasive, inexpensive measurement that is currently used to provide breath-by-breath monitoring for abrupt changes in ventilatory parameters, such as those due to pulmonary embolism, esophageal intubation, endobronchial migration of the endotracheal tube, and inadvertent extubation or disconnection of the endotracheal tube from the ventilator². It would be highly beneficial to the care of critically ill patients if PETCO₂ could also be employed as a suitable surrogate for PaCO₂. Unfortunately, in most studies, there are large and variable differences between PETCO₂ and PaCO₂ (for example, as seen in FIGS. 1 and 5 of McDonald et al.²). Even after a measurement of a baseline partial pressure gradient of CO₂ between end-tidal gas and arterial blood (PET-aCO₂), the reliability of assuming changes in PaCO₂ from serial PETCO₂ measurements does not improve.⁴

### SUMMARY OF THE INVENTION

Despite past studies showing that the differences between PETCO₂ and PaCO₂ were too large and variable to be clinically useful as a surrogate measure of PaCO₂, it has now been discovered that PETCO₂ can be used as a surrogate for PaCO₂ in ventilated mammals with lung and cardiac pathology. In particular, it has been determined that delivery, for example, end-inspiratory delivery, of a gas comprising carbon dioxide, for example a gas that has a partial pressure of carbon dioxide that simulates rebreathing, reduces (optionally minimizes) the partial pressure gradient between the patient's PETCO₂ and arterial PCO₂ (PaCO₂) to the extent that the patient's PETCO₂ becomes a clinically reliable approximation of the patient's PaCO₂. This approximation can be described as being reliable having regard to a selected “threshold of convergence” between the arterial and end tidal PCO₂ values. In one embodiment, a particular degree or threshold of convergence is determined ad hoc by delivering a carbon dioxide containing gas, optionally end inspiratory gas, to partially make up the patient's inspiratory gas volume requirement for a series of breaths, optionally until the gradient is minimized.

As discussed below, the approximate value of the PETCO₂ following delivery of CO₂ to effect the convergence between PETCO₂ and PaCO₂ values is an acceptable surrogate value of PaCO₂ for clinical purposes according to the invention since this value reflects on the current PaCO₂ within a medically acceptable margin of error (and a medical practitioner can allow the PaCO₂ to drift away from the approximated value without intervention even though it has drifted upward in the course of the subject inspiring a carbon dioxide containing gas—alternatively adjustments can be made e.g. to the frequency and tidal volume settings of the ventilator to restore a prior value). A typical upward drift in the PaCO₂ as a result of delivering CO₂ to a patient according to the invention (to cause a convergence of PaCO₂ and PetCO₂ values) can be expected to be in the order of 2-4 mm Hg, which is typically not greater than the breath to breath variation in PETCO₂. Alternatively, the PETCO₂, obtained as a surrogate measure of PaCO₂, can subsequently be adjusted by the medical practitioner to a targeted value, if needed or desired. In either case, this surrogate PaCO₂ value will have been reliably (even though somewhat artificially) ascertained and/or subsequently adjusted to predictably fall within a range of a values that are therapeutically desirable for the patient, having regard to what was determined to be an acceptable measurement error in the first place (i.e. based on a calculated error margin associated with the observed differences between the PETCO₂ and PaCO₂ values (hereafter the PET-aCO₂). The term “acceptable margin of error” means having regard to the degree of accuracy, the condition being monitored and the opportunity for intervention, a clinically useful approximation for the purpose of evaluating the need for medical intervention. This is objectively determinable according to criteria well known to a critical care physician or any predetermined consensus of medical opinion. Even for a patient in grave condition, a consistent and reliable clinical estimate within (±) 6 mm Hg of the true value may be deemed to be acceptable as a margin or error and “clinically reliable”. Optionally, predictions attainable herein within a margin or error of ±5 mmHg, for example, within a range of ±3 mmHg are attainable for embodiments of the invention.

For the purposes of the present invention, the inventors have determined that delivering a gas containing CO₂ for one or more, ideally for a series of consecutive breaths, achieves a marked convergence of PETCO₂ and PaCO₂ values. A value of PET-aCO₂ can be practically ascertained by empirically monitoring the convergence of PetCO₂ and PaCO₂ values following delivery of a standardized or titrated amount of a carbon-dioxide-containing gas. The inventors have determined that administering CO₂ for a portion of each of a series of inspiratory cycles achieves for varied purposes (i.e. a variety of pathologies constituting a form of pulmonary dysfunction) a markedly reduced difference in PETCO₂ and PaCO₂ values. The margin of error with respect to this difference is small enough to serve the practical utility of using this estimated PaCO₂ value as a reliable approximation of the value of the patient's then current PaCO₂ or as a departure point for subsequently adjusting the PaCO₂, if needed or desired, with a calculated degree of precision, by targeting and attaining a new targeted PETCO₂ according to methods known to those skilled in the art. Therefore, the inventors have determined that the “threshold of convergence” that demarcates one aspect of the invention lies in the essential principle of delivering CO₂ in an amount needed to effect a convergence in those values to an extent that approximates the value of PET-aCO₂ that is determined to be attainable in practice after experimental titration. Accordingly, a predetermined conception of what is suitable threshold of convergence for a given condition and/or type of patient can be formulated based on empirical observations yielding data on post CO₂ delivery values of PaCO₂ and the margin of error after the convergence (a PET-aCO₂ value with a calculated margin of error), optionally as a departure point to immediately adjust the approximated value of PaCO₂ when desired or needed or to adjust the amount of CO₂ delivered in the breath by adjusting the volume or concentration of CO₂ in the inspired breath to further reduce PET-aCO₂ or reduce the rise in PaCO₂ resulting from the addition of CO₂ to the breath.

Therefore, according to one aspect the invention is directed to a method of determining a value of PET-aCO₂ associated with a patient or group of patients suffering from a form of pulmonary dysfunction (optionally accompanied by a description of the margin of error associated with calculating the PET-aCO₂ value) by administering to the one or more patients for a series e.g. a plurality of consecutive inspiratory cycles, one or more gases comprising carbon dioxide. In this manner, the invention is directed to reduce or minimize the PET-aCO₂ with effect that the patient's PETCO₂ is a clinically reliable approximation of the patient's PaCO₂. For example, the invention can be readily implemented by organizing rebreathing for a plurality of inspiratory cycles.

According to one embodiment of the invention, by delivering the patient's exhaled gas for a number of breaths that is pre-determined for the condition or class of patient or determined ad hoc, the achievement of a predetermined attainable “threshold of convergence” can readily be monitored. For the purposes of the invention, achieving a reasonable and practically attainable “threshold of convergence”, however defined, supplants the need to determine an actual arterial PCO₂ value prior to effecting the convergence because knowing, for example, the average PET-aCO₂ values and an accompanying statistical measure of the average error, such as a standard deviation or standard error, allows a medical practitioner to intervene to cause this so-called surrogate PaCO₂ value to be adjusted, if needed, to a desired value within a range, with acceptable precision having regard to the targeting method and the degree of error determined to be acceptable for the surrogate measurement. Additionally, the accuracy of the value is corroborated in the process by ascertaining the precision with which a new end tidal value is obtained having regard to the precision of the targeting algorithm. Therefore the surrogate PaCO₂ value obviates the need to determine the actual PaCO₂ value through direct arterial puncture and is instead adequately represented by a statistically and clinically acceptable approximation of the true value (unknown) which, though possibly changed by the process of the invention, constitutes a precise enough instant measure of the true value for clinical evaluation or a departure point for further change of the patient's clinical management if needed.

Accordingly, in one aspect, the invention is directed to a method for determining a surrogate measure of the partial pressure of CO₂ in the arterial blood (PaCO₂) of a ventilated patient (or optionally, a spontaneously breathing patient) with pulmonary dysfunction preliminary to a diagnostic assessment of the patient's condition, comprising the step of delivering to the subject for a plurality of consecutive inspiratory cycles one or more gases comprising carbon dioxide. In this manner, the invention is directed to reduce or minimize the partial pressure gradient between the patient's PETCO₂ and PaCO₂ with effect that the patient's PETCO₂ is a clinically reliable approximation of the patient's PaCO₂. For example, the invention can be readily implemented by organizing rebreathing for a plurality of inspiratory cycles.

The term “clinically reliable approximation” means with respect to a patient's PaCO₂ means, for purposes herein, reliable for diagnostic purposes including purposes for which an invasive procedure to measure of arterial PCO₂ is warranted. Note that this term is used to describe the accuracy of predicting a PaCO₂ value from a PETCO₂ value post-administration of CO₂ to effect a convergence in those values. In a quantitative sense the phrase “clinically reliable approximation” will invariably encompass a degree of deviation from actual that constitutes an acceptable standard error for the condition of the patient under evaluation as discussed herein.

The term “pulmonary dysfunction”, for the purposes herein, broadly means pulmonary disease, for example, a disease that affects the distribution of blow flow in the lung or the distribution of ventilation in the lung or both, or systemic disease, which or the treatment of which (a direct effect or side effect of the treatment), affects the distribution of blow flow in the lung or the distribution of ventilation in the lung (or both) and includes a cardiac condition that is manifested in abnormalities of the matching of regional air flow (V) to lung perfusion (Q) and includes conditions such as reduced lung compliance, pulmonary edema, lung consolidation, and atelectasis. It is to be understood that pulmonary dysfunction at the extremes of high ventilation and low perfusion, is referred to as ‘alveolar deadspace’ or high V/Q disease. At the other extreme of low ventilation and persistent perfusion, this is referred to as ‘shunt’ or low V/Q. In addition to these extremes all abnormal lungs also exhibit intermediate states which may be due to: a) abnormal gas flow distribution (e.g. due to inflammation, secretions in the airways, bronchospasm, changes in regional lung compliance) and increases in the diffusion barriers at the alveoli (e.g. pulmonary edema, pneumonia) and b) changes in lung perfusion (e.g. due to pulmonary embolism, pulmonary artery hypertension, pulmonary artery hypotension, regional increases in blood flow due to inflammation, decreases in blood flow due to regional increases in resistance such as due to hypoxic pulmonary vasoconstriction). Additionally, cardiac shunting of blood between pulmonary arterial and venous circulations is also encompassed by the term “pulmonary dysfunction” as used herein as such conditions also affect the PET-aCO₂. Cardiac shunting is classified as left-to-right shunts, for example ventricular septal defect, atrial septal defect, patent foramen ovale, and right-to-left shunting such as patent ductus arteriosus, atrial septal defect and patent foramen ovale in the presence of increased pulmonary artery pressure.

In another aspect, the invention is directed to the use of a gas delivery system, optionally comprising a ventilator, to determine arterial blood gas concentrations in a (optionally) ventilated patient with pulmonary dysfunction, the gas delivery system organized to deliver to the patient for a plurality of consecutive inspiratory cycles one more gases comprising carbon dioxide in an amount sufficient to minimize the partial pressure gradient between the patient's PETCO₂ and PaCO₂ whereby the patient's PETCO₂ is a clinically reliable approximation of the patient's PaCO₂.

A gas delivery system according to the invention is a respiratory gas delivery system which comprises an airflow control system. The airflow control system may be connected between a gas source, for example, a source of driven gas, for example a ventilator; or an anesthetic machine (which may include a ventilator), and a set of gas conduits leading to a patient airway interface (typically a mask or endotracheal tube) including means to control the flow of gas such as one or more valves. Optionally, the airflow control system comprises or is connected to an expiratory limb and an inspiratory limb which may, in turn connect to the patient airway interface, for example via a Y piece. The inspiratory and expiratory limbs are connected to or comprise portions connectable to the Y piece and portions connected to the ventilator. A port or other device for introducing a carbon dioxide containing gas directly or indirectly (via a limb of a breathing circuit) into the patient airway interface (hereafter broadly referred to an equalizer) may optionally be interposed between the two aforesaid portions of the inspiratory limb and the two aforesaid portions of the expiratory limb, for example, in one embodiment to divert airflow directed from the ventilator, optionally in the course of a single inspiratory cycle, from the inspiratory limb to the expiratory limb, for one or more inspiratory cycles. For example, in this manner, airflow initially channeled to the Y piece via the inspiratory limb is diverted and channeled to the Y piece via the expiratory limb so that gas residing in the portion of the expiratory limb proximal to the patient airway interface may be driven by the ventilator to the patient airway interface.

In another aspect, the invention is directed to a ventilator comprising a carbon dioxide delivery system adapted to deliver to the patient for a plurality of consecutive inspiratory cycles one or more gases comprising carbon dioxide in an amount sufficient to minimize the partial pressure gradient between the patient's PETCO₂ and arterial PaCO₂ whereby the patient's PETCO₂ is a clinically reliable approximation of the patient's PaCO₂.

Optionally, the carbon dioxide delivery system comprises an airflow control system for channeling or otherwise organizing airflow from a primary inspiratory gas source (or route) to an alternative inspiratory gas source comprising carbon dioxide, wherein the primary and alternative gas sources/routes collectively deliver to the patient for a plurality of consecutive inspiratory cycles one more gases comprising carbon dioxide in an amount sufficient to reduce or minimize the partial pressure gradient between the patient's PETCO₂ and PaCO₂ such that the patient's PETCO₂ is a clinically reliable approximation of the patient's PaCO₂.

In another aspect, the invention is directed to a breathing circuit for use in conjunction with a ventilator comprising:
an expiratory limb for establishing a fluidic connection between the ventilator and a patient airway interface;
an inspiratory limb for establishing a fluidic connection between the ventilator and a patient airway interface;
an airflow control system for channeling airflow from the ventilator to one of the limbs for any first portion of an inspiratory cycle and diverting airflow generated by the ventilator to the other limb during any second portion of an inspiratory cycle, and wherein airflow diverted via the expiratory limb delivers exhaled gas stored in the expiratory limb in an amount (per breath) sufficient to reduce or minimize the partial pressure gradient between the patient's PETCO₂ and PaCO₂ such that the patient's PETCO₂ is a clinically reliable approximation of the patient's PaCO₂.

Optionally, the expiratory limb includes a expiratory gas reservoir portion proximal to the patient airway interface and wherein the airflow control system is interposed between the ventilator and the expiratory gas reservoir portion of the expiratory limb for driving expiratory gas contained in the expiratory gas reservoir portion towards the patient airway interface during one of the first or second portions of an inspiratory cycle.

Optionally, the airflow control system comprises a valve including at least one airflow channel segment that is fluidically connectable to the ventilator (this airflow channel optionally comprises an inspiratory ventilator portion and an expiratory ventilator portion) at least one inspiratory airflow channel segment (alternatively referred to as an inspiratory segment or inspiratory limb portion) that is fluidically connectable to the inspiratory limb, at least one expiratory airflow channel segment (alternatively referred to as an expiratory segment or expiratory limb portion) that is fluidically connectable to the expiratory limb and at least one airflow closure portion (alternatively referred to as an airway blocking member, airway closure or airway closure member), the at least one airflow closure portion operatively associated with the least one inspiratory airflow channel segment and the at least one expiratory airflow channel segment for reversibly opening and closing the respective segments alternately. Optionally at least one airflow channel closure portion is movable between an inspiratory airflow channel segment occluding position (alternatively referred to as an inspiratory segment occluding position or inspiratory limb occluding position) and an expiratory airflow channel segment occluding position (alternatively referred to as an expiratory segment occluding position or expiratory limb occluding position). Optionally, the at least one airflow channel closure portion is pressure responsive or time responsive or moves in synchronization with ventilator and is thereby optionally synchronized with different respective portions or phases of an inspiratory cycle to deliver carbon dioxide e.g. previously exhaled gas, in at least one such portion or phase. Optionally, the airflow channel closure portion is an airflow blocking member, optionally in the form of a shuttle member. Optionally the shuttle member, analogous to and/or operating as a valve flap/closure or valve plate, is operatively associated with two valve seats, alternatively sitting on one valve seat and then the other. Optionally, the airflow blocking member e.g. a valve plate is, or is operatively coordinated to the phase of inspiration, for example associated with an air pressure responsive member (e.g. a separate member). Optionally, the airflow blocking member is biased to occupy the expiratory airflow channel segment occluding position. Optionally, the airflow blocking member is adapted to move in opposition to a biasing force in response to an increase in air pressure towards the end of inspiration. Optionally, a separate air pressure responsive member operatively connected to the airflow blocking member (e.g. connected for linear movement therewith e.g. via a rod) acts against a biasing force responsive to an increase in air pressure at the latter part of inspiration so that the airflow blocking member assumes the inspiratory airflow channel segment occluding position. Optionally, a biasing element e.g. in the form of a magnet or a spring acts on the airflow blocking member to bias the airflow blocking member into the expiratory airflow channel segment occluding position.

Alternatively, the at least first airflow channel segment and the at least a second airflow channel segment are each operatively associated with a dedicated airflow channel closure portion. Optionally the dedicated airflow channel closure portions are operatively associated for coordinated movement.

In yet another aspect the invention is directed to a valve for use in conjunction with a ventilator breathing circuit of the type having an inspiratory limb for establishing a fluidic connection between the ventilator and a patient airway interface and an expiratory limb establishing a fluidic connection between the ventilator and the patient airway interface, the valve adapted to redirect ventilator flow from the inspiratory limb to an expiratory gas reservoir portion of the expiratory limb during inspiration to drive exhaled gas residing in the expiratory limb towards the patient airway interface during an inspiratory cycle.

In yet another aspect, the invention is directed to the use of a gas delivery system to determine an arterial blood gas concentration in a patient with pulmonary dysfunction, the gas delivery system organized to deliver to the patient, for at least one inspiratory cycle, optionally for a series, optionally a plurality of consecutive inspiratory cycles, one more gases comprising carbon dioxide to diminish or minimize the partial pressure gradient between the patient's PETCO₂ and arterial PCO₂. Optionally, the gas comprising carbon dioxide is a gas that has partial pressure of carbon dioxide that simulates (the partial pressure of) or is constituted in whole or part by the patient's previously exhaled gas.

Optionally, the use comprises the step of ascertaining the value of PETCO₂ at the end a plurality of inspiratory cycles.

Optionally, the gas delivery system is organized to deliver a first gas, for example a gas that matches the patient's respiratory needs, for at least a portion of each inspiratory cycle and a second gas comprising or constituted by the patient's exhaled gas (or a gas that approximates the carbon dioxide content of the exhaled gas), is delivered for at least a portion of each inspiratory cycle. Optionally, the gas exhaled at the end the immediately preceding inspiratory cycle, is delivered for at least a portion, optionally a different portion, of each inspiratory cycle.

Optionally, a value of PETCO₂ is ascertained at the end of one or more of a plurality of inspiratory cycles and one such value, a value that meets a threshold of convergence as herein defined, is ascertain for later use to make a diagnostic evaluation of the patient's condition.

In yet another aspect, the invention is directed to the use of a CO₂ delivery system, breathing circuit or a valve to diminish the partial pressure gradient between measured PETCO₂ values and actual PaCO₂ values in patients with pulmonary dysfunction, the use comprising the steps of delivering a gas comprising CO₂ for one or more, ideally for a series of breaths, for example a plurality of consecutive inspiratory cycles and then ascertaining a PETCO₂ value for the last such breath e.g in the course of the expiratory cycle immediately following the last such inspiratory cycle (in the case of one breath during the course of an expiratory cycle after the breath). Optionally, the amount of CO₂ delivered for each of several consecutive inspiratory cycles modifies the partial pressure of CO₂ in the inspiratory gas to a pre-selected or empirically determined value, for example, to achieve a fractional concentration of CO₂ that is or approximates 6% or for example, a value that approximates the PETCO₂ in a immediately preceding expiratory cycle. Optionally, the CO₂ delivery system, breathing circuit and/or valve makes exhaled gas, optionally, the end tidal gas of respective expiratory cycles immediately preceding each of a series of inspiratory cycles, available for inspiration during the course of the respective inspiratory cycles, for example for a portion, optionally the last portion, of each respective inspiratory cycle. Optionally, the valve is a rebreathing valve, for example a sequential gas delivery (SGD) valve (i.e. a valve that timed to open (e.g. at a particular juncture in an inspiratory portion of a ventilation cycle) and/or responsive to open at a predetermined airway pressure to deliver a second gas (e.g. comprising CO₂) during the course of each inspiratory cycle, in sequence, for example before or after delivery of a first gas, the first gas optionally delivered for a first portion of each inspiratory cycle—for example after depleting in each such inspiratory cycle, a gas reservoir containing the first gas, the circuit organized such that an increase in airway pressure impinges on a valve set to open at that increased pressure (a re-breathing valve, optionally in the form of an SGD valve) after depletion of the first gas reservoir allows passage of a second gas to a patient airway interface e.g. exhaled gas, optionally end tidal gas, stored in the circuit, usually in an expiratory limb of the circuit which may be a conduit segment or bag, for example, a dedicated expiratory gas reservoir. Optionally, in the case of a gas delivery system, breathing circuit or valve, operatively associated with a ventilator for a non-spontaneously breathing (ventilated) patient (as opposed to a spontaneously breathing patient) a valve is adapted to repeatedly redirect flow from the ventilator towards a reservoir of exhaled gas to make the exhaled gas available for inspiration during at least a portion of the inspiratory cycle, optionally the latter part of each such cycle e.g. by driving the exhaled gas towards to a patient airway interface e.g. an interface formed together with or connected to a Y-piece that receives the primary inspiratory flow through an inspiratory branch of the Y-piece and the exhaled gas via a re-directed flow to an expiratory branch of the Y-piece.

In yet another aspect, the invention is directed to a breathing circuit for use in conjunction with a ventilator comprising:

Means (optionally constituted by or comprising a conduit) defining an expiratory gas airflow pathway;
Means (optionally constituted by or comprising a conduit) defining an inspiratory gas airflow pathway;
Means for comingling (optionally channeling via a conduit and/or valve) inspiratory gas and expiratory gas in an amount sufficient to equilibrate the patient's PETCO₂ and arterial PCO₂ such that the patient's PETCO₂ is a clinically reliable approximation of the patient's PaCO₂, optionally by channeling expiratory gas into the inspiratory gas, optionally in the inspiratory gas flow pathway. For example, a suitably sized bridging portion or conduit may be sued to connect the expiratory limb to the inspiratory limb.

### BRIEF DESCRIPTION OF THE DRAWINGS

FIG. 1 is a schematic representation of a breathing circuit according one embodiment of the invention illustrating the principle of operation of the airflow control system of the breathing circuit;

FIG. 2 is a schematic representation of a valve according to one embodiment of the invention illustrating the operation of the valve during expiration;

FIG. 3 is a schematic representation of a valve according to one embodiment of the invention illustrating the operation of the valve during of a breathing first portion of the inspiratory cycle;

FIG. 4 is a schematic representation of a valve according to one embodiment of the invention illustrating the operation of the valve during a second portion of the inspiratory cycle.

FIG. 5 is a schematic representation of an improvised breathing circuit that can be applied to most ventilatory circuits (including anesthesia circuits) and adjusted to induce rebreathing at the end of the breath. FIG. 5a shows an airflow control system according to one embodiment of the invention illustrating the condition of a circuit during the first part of inspiration. FIG. 5b shows an airflow control system according to one embodiment of the invention illustrating the condition of a circuit during the second or later part of inspiration.

FIG. 6 is a table (Table 1) presenting data related to differences between measured PetCO₂ and PaCO₂ values derived from the study described in Example 1.

FIGS. 7a through 7f illustrate divergence in PETCO₂ and PaCO₂ values in prior art studies.

FIG. 8 is a Table itemizing the conditions of the piglets used in the study described in Example 1.

FIG. 9 illustrates a breathing circuit including a ventilator that may be adapted for implementation of the invention.

FIG. 10 illustrates two Bland Altman plots that are used to compare results obtained from Example 1 (Panel A) with duplicate arterial puncture values (Panel B).

FIG. 11 is a graphical representation of data obtained from Example 1 in the form of a Bland Altman plot.

### DETAILED DESCRIPTION OF THE INVENTION

The present invention based on the discovery that end-inspiratory delivery of a gas comprising carbon dioxide (for example a gas that has a partial pressure of carbon dioxide that simulates carbon dioxide intake associated with rebreathing exhaled gas), in ventilated patients with pulmonary dysfunction, reduces the partial pressure gradient between the patient's PETCO₂ and PaCO₂ to the extent that the patient's PETCO₂ becomes a better approximation of the patient's PaCO₂. This reduction in partial pressure gradient may serve diagnostic purposes in patients that are ventilated due to abnormal regional or global gas flow distribution due to inflammation, bronchospasm and increased secretions in the airways such as due to asthma, allergy, bronchitis, pneumonia, autoimmune bronchitis and alveolitis, inhalation of toxic or caustic vapors or liquids, aspiration of stomach contents, systemic effects of sepsis, liver failure, renal failure; changes in regional lung compliance due to lung edema (of various etiology such as infection, heart failure, trauma, exposure to caustic and irritant gases and liquids, fibrosis, in combination these are known as adult respiratory distress syndrome (ARDS); increases in the lung diffusion barriers at the alveoli due to pulmonary edema, pneumonia, fibrosis, infiltration with cancer cells; changes in lung perfusion due to changes in pulmonary artery pressure, shunting of blood in the heart or ductus arteriosus, obliteration of alveolar capillaries, or blood clots such as pulmonary embolism, increased pulmonary artery pressure, pulmonary hypertension, pulmonary artery hypotension, regional increases in blood flow due to inflammation, decreases in blood flow due to regional increases in resistance such as due to hypoxic pulmonary vasoconstriction.

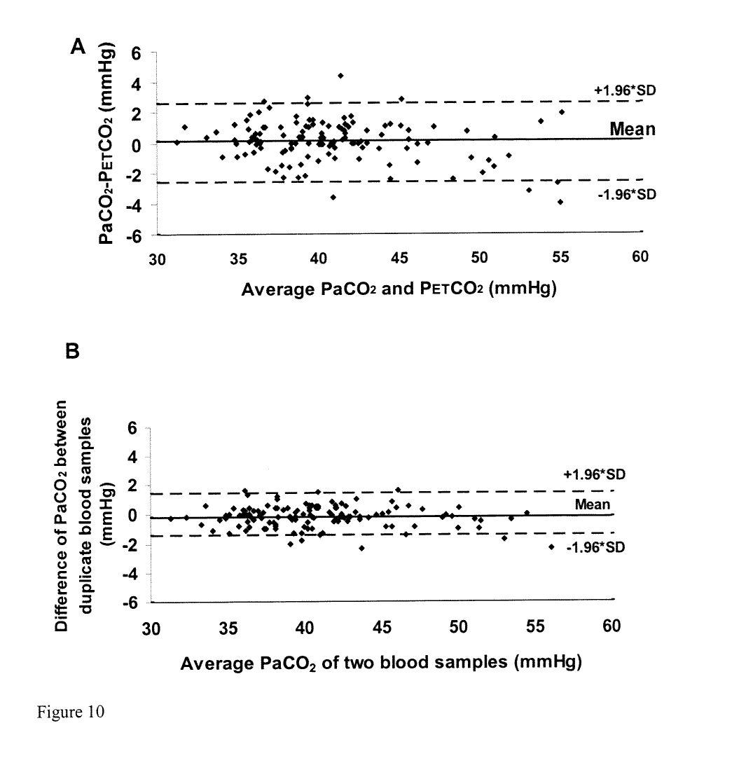

Values of PET-aCO₂ and acceptable margins of error for purposes of the invention are attainable according to the invention by delivering CO₂ to effect a convergence in PaCO₂ and PETCO₂ values. Gancel (Gancel P E, Roupie E, Guittet L, Laplume S, Terzi N. Accuracy of a transcutaneous carbon dioxide pressure monitoring device in emergency room patients with acute respiratory failure. Intensive Care Med 2010 November 11.) had indicated that with a low bias (0.1 mmHg), the limits of agreement ranging from −6.0 to 6.2 mmHg “was clinically acceptable”. The aforementioned study by Gancel et al. investigated a transcutaneous PCO₂ measurement as a surrogate for PaCO₂. They deemed the ±6.0 mmHg difference between the trans-cutaneous PCO₂ and PaCO₂ “clinically acceptable”. Such low ranges are seldom obtainable in PET-aCO₂ and certainly cannot be expected to be predictably obtainable as a rule without the present invention. In studies performed by the inventors in sick adult pigs with severe lung atelectasis and pneumonia the PETCO₂ and the PaCO₂ were statistically indistinguishable over a wide range of PETCO₂ and oxygen levels, with the average PET-aCO₂ (mean±SE) of −0.13±0.12, 95% Cl: −0.36, 0.10 (p=0.3). The inventors found that PET-aCO₂ (FIG. 10A) did not differ from the difference in PaCO₂ between duplicate arterial blood samples (FIG. 10B) (−0.19±0.06 mmHg, 95% Cl: −0.32, −0.06) (p=0.66) indicating that the PET-aCO₂ was the same as the difference between two consecutive invasive blood gas analysis. Thus not only was PETCO₂ a precise surrogate for PaCO₂, but was no worse than an invasive blood gas measurement at measuring PaCO₂. A surrogate value of PaCO₂ obtained according to the invention herein is considered a “clinically reliable approximation” or one involving an “acceptable margin of error”. For purposes herein, this means reliable for diagnostic purposes including purposes for which an invasive procedure to measure of arterial PCO₂ is warranted. Note that this term is used to describe the accuracy of predicting a PaCO₂ value from a PETCO₂ value post-administration of CO₂ to effect a convergence in those values. In a quantitative sense the phrase “clinically reliable approximation” will invariably encompass a degree of deviation from actual that constitutes an acceptable standard error for the condition of the patient under evaluation. As a bench mark for a grave condition we note that the above-mentioned criteria of Gancel et al. (Gancel P E, Roupie E, Guittet L, Laplume S, Terzi N. Accuracy of a transcutaneous carbon dioxide pressure monitoring device in emergency room patients with acute respiratory failure. Intensive Care Med 2010 November 11) were established with respect to emergency room patients with acute respiratory failure. According to the invention as herein defined “clinically acceptable” for the purpose of defining an acceptable margin of error means reliably no less accurate than +/−6.0 mm Hg and what the inventors found that the delivering CO₂ to effect a convergence of PaCO₂ and PetCO₂ values reliably surpasses this standard.

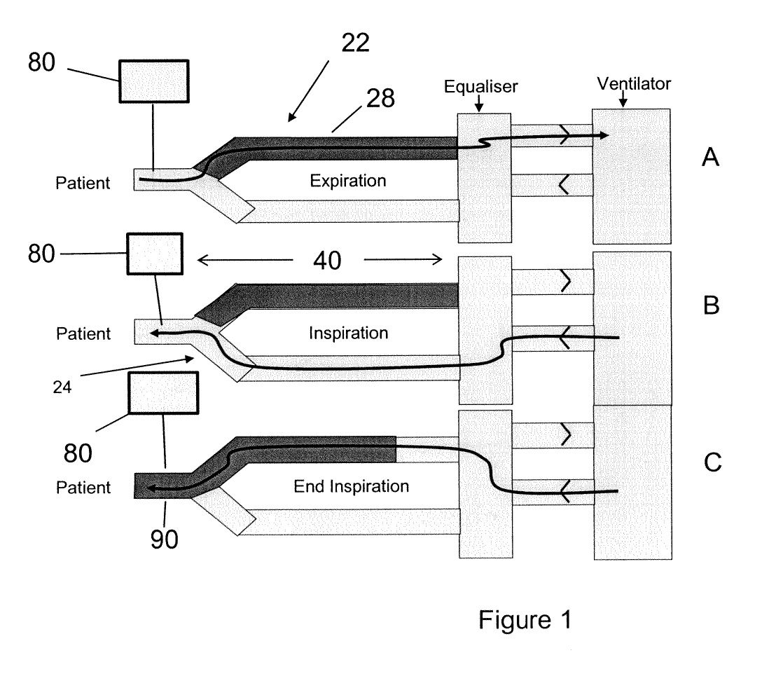

According to one embodiment of the invention, a gas delivery system according to the invention functions in the manner schematically illustrated in FIG. 1. The term “equalizer” is coined to refer to a device that delivers a gas comprising carbon dioxide to the patient for a portion of each of a plurality of consecutive inspiratory cycles to minimize the partial pressure gradient between the patient's PETCO₂ (end tidal partial pressure of CO₂) and PaCO₂ whereby the patient's PETCO₂ is a clinically reliable approximation of the patient's PaCO₂. According to one embodiment of the invention the device is operatively associated with or part of a breathing circuit in a manner that channels airflow from the ventilator to one of the limbs for a first portion of an inspiratory cycle and diverts airflow generated by the ventilator to the other limb of the circuit (a limb housing expired gas) during any second portion of an inspiratory cycle, in order to deliver the patient's expired gas to the patient. Optionally, the gas delivery system employs a means, for example a valve, that is controlled (e.g. mechanically based on a set opening pressure or via a controller) to combine the flow of two gases or alternate flow repeatedly between a first gas, for example a gas that closely matches the patient's respiratory requirements (a principal inspiratory gas), and a gas comprising CO₂, for example, as a result of being set to cycle based on time, or based on a pre-determined volume of inspired gas, or based on being synchronized to a ventilator. The term “subject” and “patient” are used interchangeably. Arrows indicate the direction and path of air flow to and from the ventilator through the equalizer.

FIG. 1 comprises Panel A showing airflow during the expiratory portion of a breathing cycle, Panel B showing airflow during the first part of the inspiratory portion of a breathing cycle, and Panel C showing airflow during a second part of inspiration. As shown in FIG. 1, at the end of expiration, expired gas (dark shaded area) remains in the expiratory tubing of the expiratory limb 22 after each expiration (A). In one embodiment of the invention, a length of expiratory tubing 28 that holds expired gas constitutes an expiratory gas reservoir portion 40 of the expiratory limb 22. During initial inspiration (B) the ventilator 20 blocks the movement of expired gas from the expiratory gas reservoir portion 40 of the expiratory limb/tubing 28 and inspiratory gas flows into the patient via the inspiratory tubing 32 of the inspiratory limb 24 (labeled in B). Airway pressure increases during the course of inspiration. In one embodiment of the invention, the equalizer 10 is represented by a breathing circuit or portion thereof that includes an airflow control system. The airflow control system 30 operates such that an airflow blocking member, optionally part of a valve, switches at a set (adjustable) time or pressure to route the inspiratory flow of gas from the ventilator to the subject/patient via the expiratory tubing 28 (C), pushing the previously expired gas in the tubing ahead of it into the subject/patient's lungs (rebreathing). This is shown in (C) by an arrow and the shaded area moved towards the patient. A suitable capnographic instrument 80 for determining the partial pressure of carbon dioxide in the patient's end tidal exhaled gas can then be used as a surrogate measure of the arterial partial pressure of CO2.

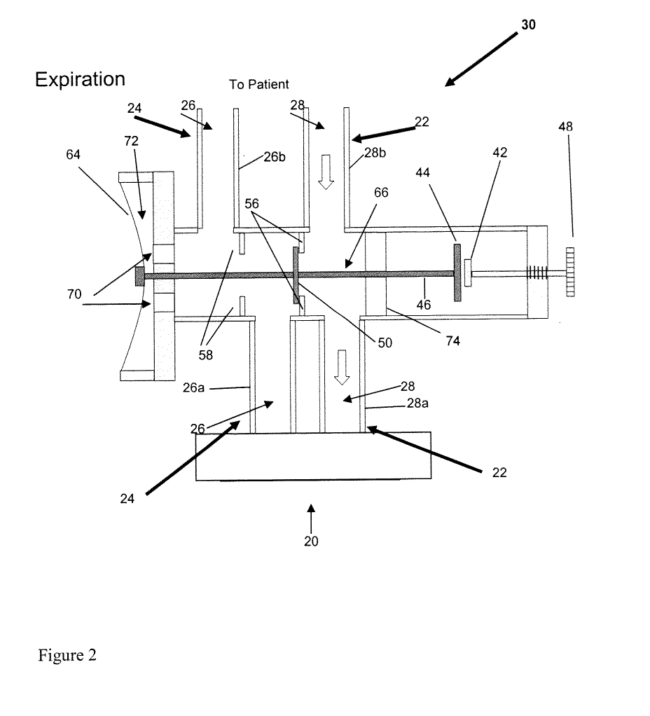

FIGS. 2, 3 and 4 show the design of an equalizer 10 comprising an airflow control system 30 optionally including components of a breathing circuit comprising an airflow control system according to one embodiment of the invention wherein the equalizer operates as or is in the form of a valve, for example a valve that can be used with conventional breathing circuits used in ventilating patients, for example a valve that can be interposed in the breathing circuit even while in use in a ventilated patient, for example such valve switching the direction of gas flow from the inspiratory to the expiratory limb when a circuit pressure threshold is reached, and re-establishing its previous configuration during the expiratory phase of the ventilator. The nature of the valve can vary. Optionally, the valve switches flow as a result of cycling based on time, or based on a pre-determined volume of inspired gas, or based on being synchronized to a ventilator.

According to one embodiment, as shown in FIG. 2, the valve comprises an inspiratory ventilator portion 26a and inspiratory limb portion 26b constituting a first airway 26 fluidically connectable between the ventilator and the inspiratory limb and an expiratory ventilator portion 28a and expiratory limb portion 28b constituting a second airway 28 fluidically connectable between the ventilator 20 and the expiratory limb 22 and at least one air flow blocking member 50 movable between a first airway occluding position and a second airway occluding position. Optionally, the valve comprises at least one biasing element in the form of magnet 42 for biasing the airflow blocking member 50 towards the second airway occluding position during the first portion of each inspiratory cycle. The position of the magnet 42 relative to a metal plate 44 mounted on rod 46 (supported, in part, by a channel in fixed plate 74) may be controlled by a screw 48. The locations of the magnet 42 and plate 44 may be interchanged. The airflow blocking member 50 is driven towards the first airway occluding position in response to an increase in airway pressure in the inspiratory limb. According to the embodiment illustrated in FIG. 2, magnet 42 is used as a biasing element to form a latch switch so that the valve changes position from valve seat 56 to valve seat 58 only after a set pressure acting on a pressure responsive member, for example a diaphragm 54 is exceeded. Alternatively, the position can be set to change based on time or a ventilator setting. Attraction of the magnet returns the airflow closure member 50 to its resting (expiratory) position. Alternatively, a spring or gravity may be used to return a closure member to an expiratory limb occluding position.

FIG. 2 illustrates one embodiment of an equalizer in the form of a valve during expiration. Wide arrows show direction of air flow. The biasing element in the form of magnet 42 operatively associated with a metal plate 44 hold an airflow blocking member 50 against valve seat 56. Thus expired flow travels along the expiratory limb 22 to the ventilator 20. Circuit pressure is expiratory pressure as set by the ventilator 20.

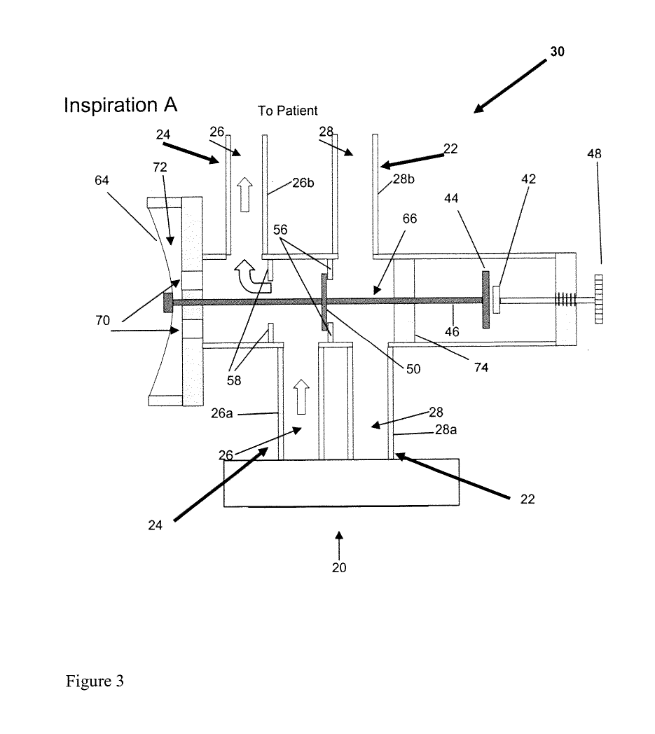

As shown in FIG. 3, the valve comprises an air pressure responsive member 64 which is operatively connected, for example via a shaft 46 or rod, to the airflow blocking member 50 and also movable between a first airway occluding position and a second airway occluding position. For example, the air pressure responsive member 64 may be positioned to oppose the action of the biasing element 42 in response to an airway pressure rise in inspiratory limb 24 during initial inspiration. For example, the air pressure responsive member may take the form of a diaphragm 64 which is in fluid communication with the first airway, optionally through pressure vents 70 leading to a chamber 72 under the diaphragm 64. As long as airway pressure in the inspiratory limb 24 and in the chamber 72 covered by the diaphragm 64 is insufficient to overcome the magnetic force holding the metal plate 44 against the magnet 42, the ventilator output enters patient inspiratory limb 24.

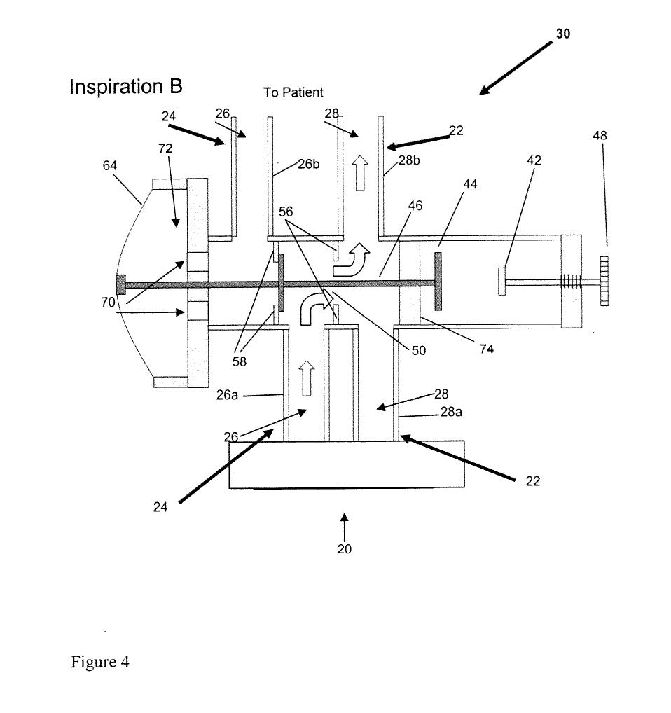

As seen in FIG. 4, as airway pressure rises during inspiration, the pressure under the diaphragm 64 increases to a threshold after which it causes the diaphragm to bow out from convex to concave shape, and displace the shaft 46, separating the metal plate 44 from the magnet 42 and shifting the valve plate 50 from valve seat 56 to valve seat 58. Inspiratory gas is then directed down the expiratory limb 22 to the patient pushing previously exhaled gas ahead of it.

Note that the airway pressure in the inspiratory limb 24 also continues to climb as the inspiratory and expiratory limbs are connected by a Y-piece 90 at the patient airway interface (e.g. an endotracheal tube—not shown). At the end of inspiratory phase of the ventilator, the airway pressure is reduced for exhalation. This reduces the pressure on the circuit side of the diaphragm 64. The attraction of the magnet 42 for the metal plate 44 resets the valve plate 50 against valve seat 56 and the expiratory configuration is re-established.

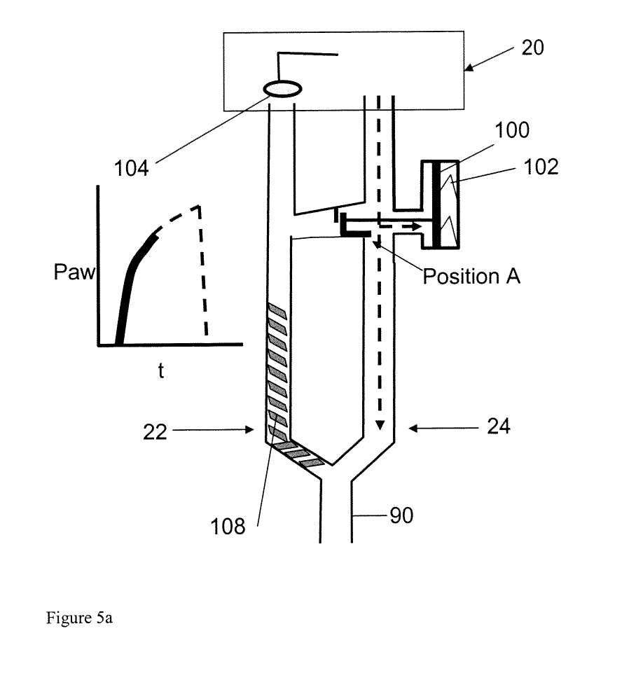

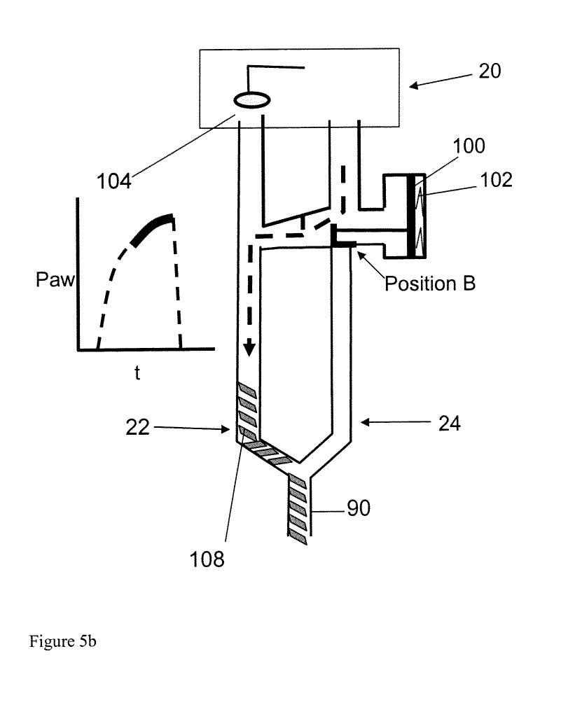

FIG. 5 illustrates a device according to one embodiment of the invention in which an equalizer in the form of an air flow control system is interposed into a standard ventilator circuit to passively implement rebreathing at end-inspiration.

As seen in FIG. 5a airway pressure rises during inspiration (FIG. 5a). During inspiration, the airway pressure (Paw) rises in the inspiratory limb 24, simultaneously pressurizing the piston 100. Before the Paw reaches a threshold valve if it is biased into Position A in which it occludes the expiratory limb. When the Paw reaches a threshold value, the piston collapses the spring(s) 102 and pulls airflow closure member in the form of a shuttle member 106 into Position B to occlude the inspiratory limb 24 and direct the inspiratory gas down the expiratory limb 22 (FIG. 5b). The gas in the expiratory limb 22 contains exhaled gas 108 (hatched) which is displaced into the patient's lung (hence rebreathing). During exhalation, an airflow blocking member, optionally in form of a mushroom valve 104 is collapsed and spring(s) 102 recoils to re-establish the position shown in FIG. 5a. The spring 102 can be bi-stable or magnets can be incorporated to achieve the same effect.

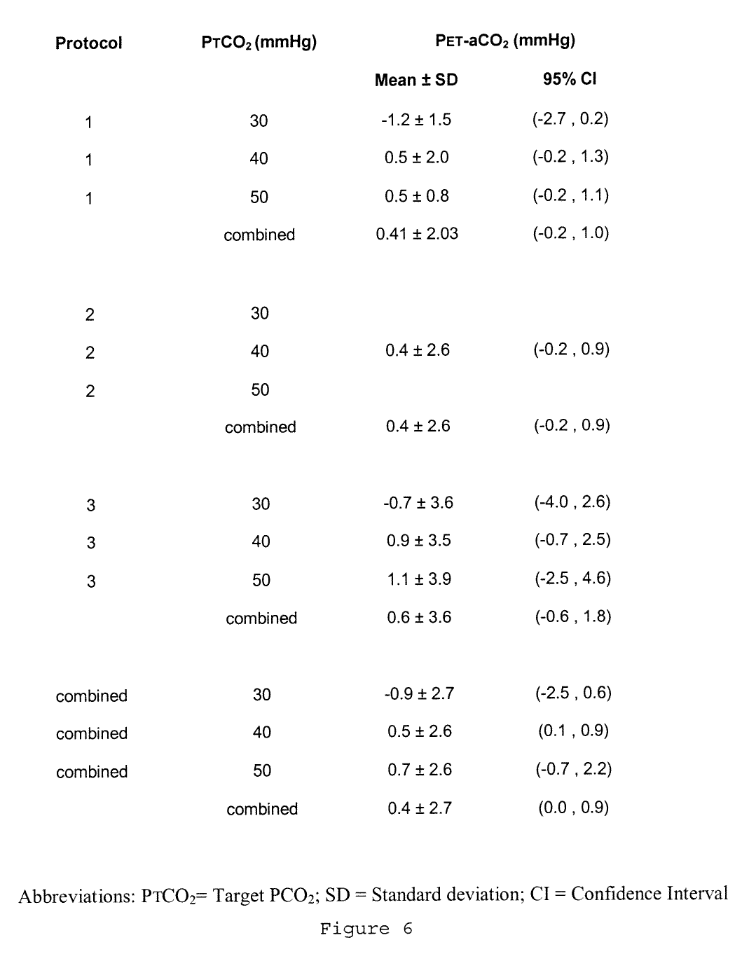

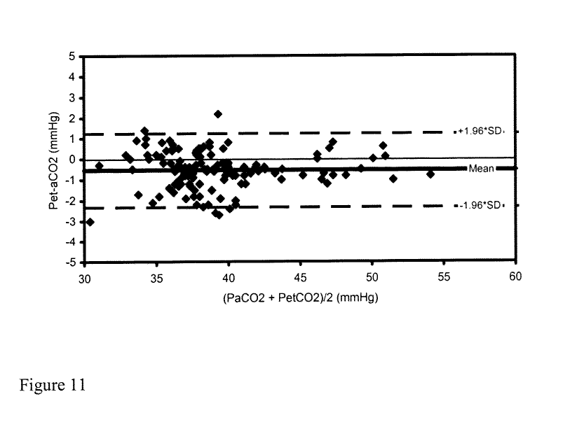

FIG. 6 shows the results of a study with eight newborn Yorkshire pigs with various combinations of acquired viral pneumonia, persistent patent ductus arteriosus, and patent foramen ovale were mechanically ventilated via a partial rebreathing circuit to implement end-inspiratory rebreathing. Arterial blood was sampled from an indwelling arterial catheter and tested for PaCO₂. A variety of alveolar ventilations resulting in different combinations of end-tidal PCO₂ (30 to 50 mmHg) and PO₂ (35 to 500 mmHg) were tested for differences between PETCO₂ and PaCO₂ (PET-aCO₂). The PET-aCO₂ of all samples was (mean±1.95SD) 0.4±2.7 mmHg. The agreement between PETCO₂ and PaCO₂ is shown in the FIG. 11 below.

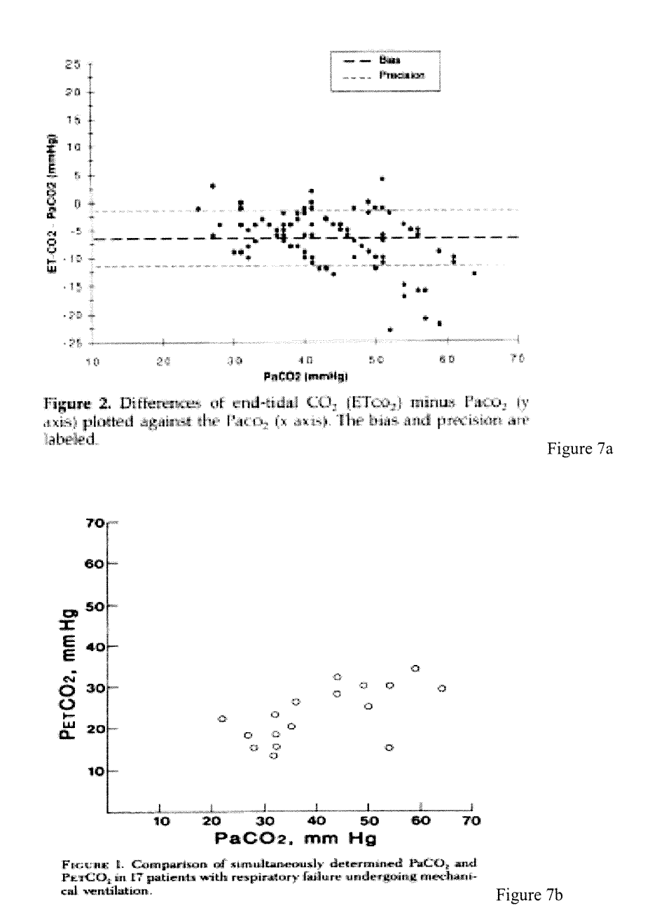

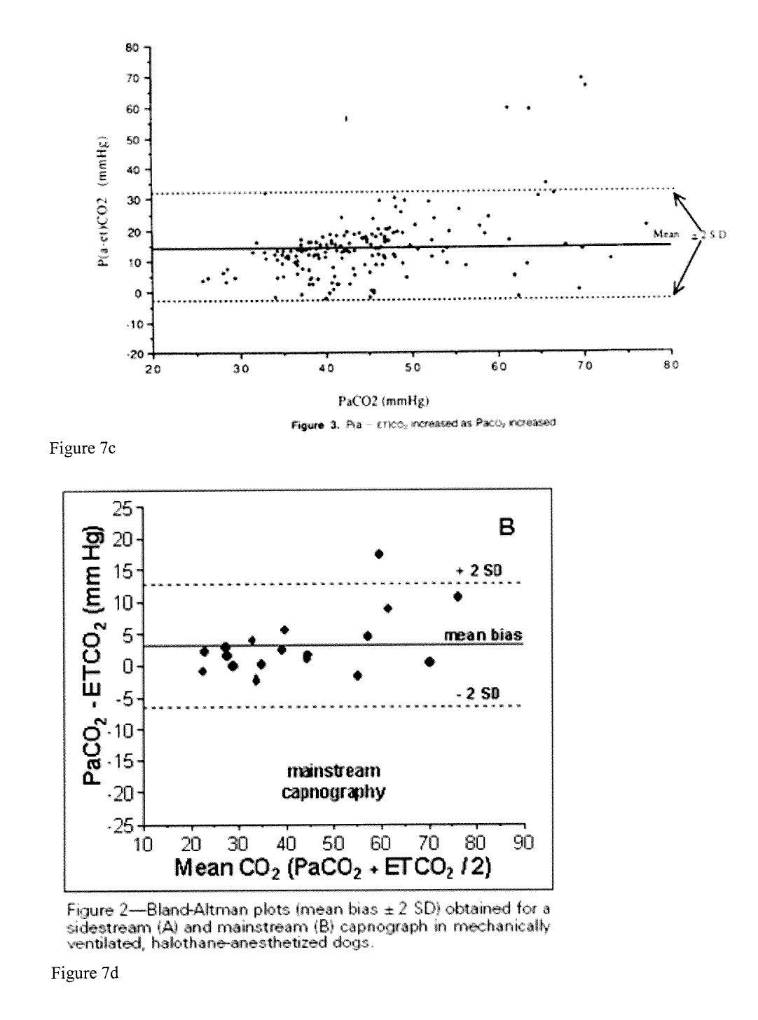

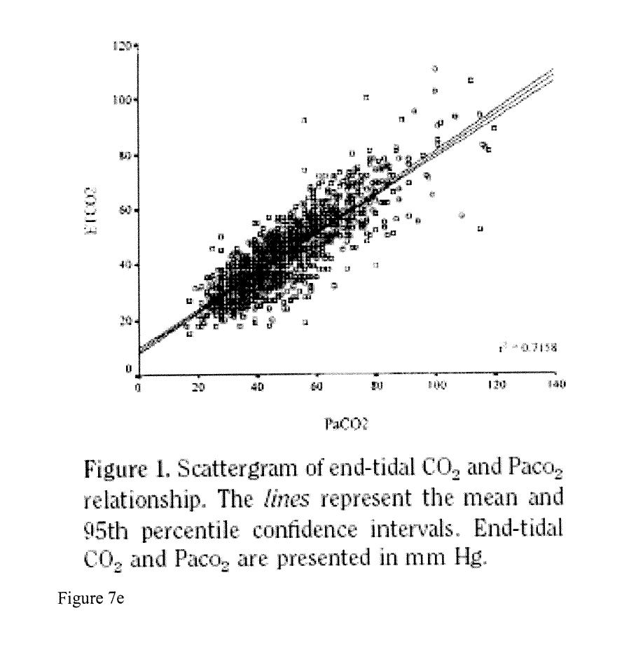

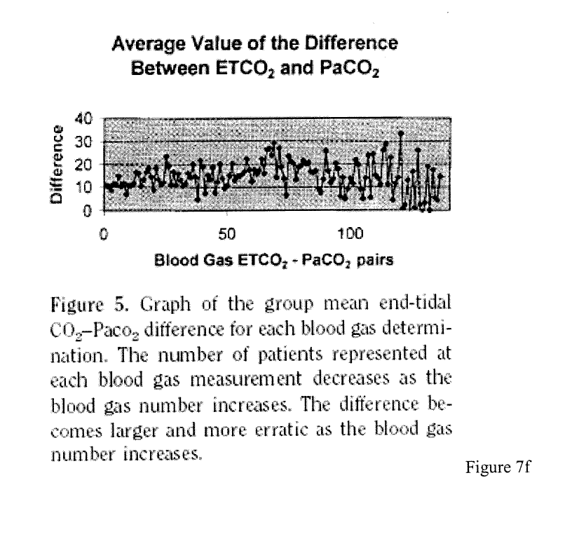

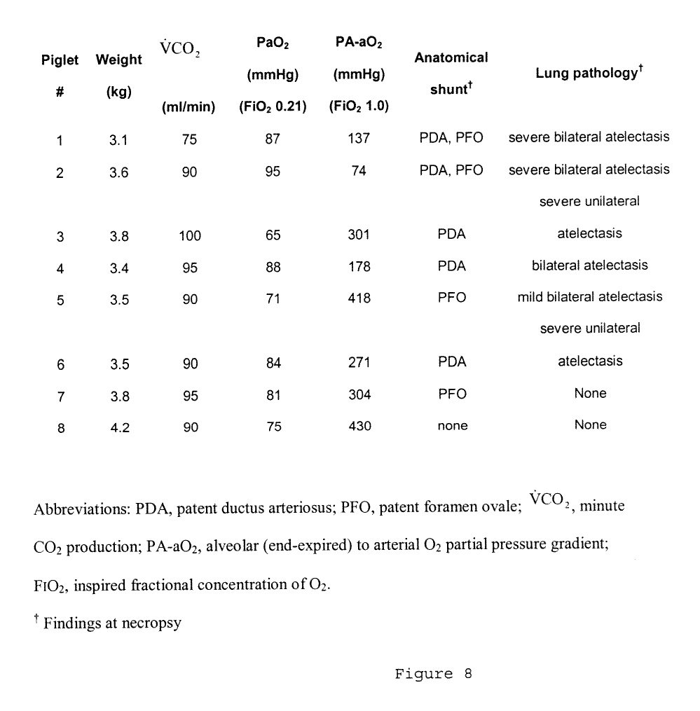

FIGS. 7a to 7f show the results of agreement between PETCO₂ and PaCO₂ from 6 studies taken from the literature. Each shows that the gradients are at least an order of magnitude greater than we were able to achieve in our animal model that had comparable lung pathology.

Methods of targeting end tidal concentrations of gases, for example to alter a surrogate measure of PaCO2 (e.g. post CO2 gas delivery and convergence of end tidal and PaCO₂ values) are described in WO/2007/012197 and in Slessarev M, et al. Prospective targeting and control of end-tidal CO₂ and O₂ concentrations J. Physiol. 2007 Jun. 15; 581 the disclosures of which are hereby incorporated by reference.

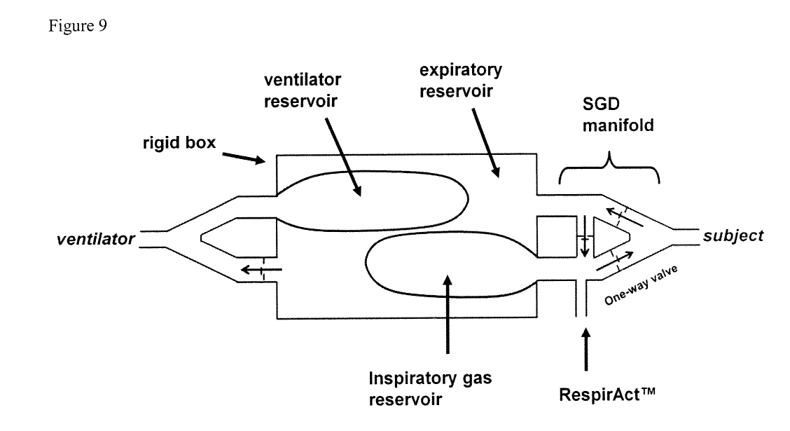

FIG. 9 illustrates an alternative circuit for testing the invention. To enable sequential gas delivery during mechanical ventilation the inventors placed a sequential gas delivery (SGD) circuit similar to that used for spontaneous ventilation (see FIG. 7 of published US patent application 2002/0185129) in a rigid container to form a functional “bag in box” secondary circuit (FIG. 9). The assembly was then interposed between the ventilator and the animal's endotracheal tube. This circuit functioned as that described by Slessarev et al. (7) with the ventilator displacing gas from the reservoir bags and the valves acting passively to provide the gas from the gas blender first, followed by the rebreathed gas.

### Example 1

Study Subjects: 8 Yorkshire newborn pigs, 3-4 weeks of age with a mean weight of 3.6 kg (table 1) in an animal operating room setting. Eight newborn Yorkshire pigs with various combinations of acquired viral pneumonia, persistent patent ductus arteriosus, and patent foramen ovale were mechanically ventilated via a partial rebreathing circuit to implement end-inspiratory rebreathing. Arterial blood was sampled from an indwelling arterial catheter and tested for PaCO₂. A variety of alveolar ventilations resulting in different combinations of end-tidal PCO₂ (30 to 50 mmHg) and PO₂ (35 to 500 mmHg) were tested for differences between PETCO₂ and PaCO₂ (PET-aCO₂).

Results: The PET-aCO₂ of all samples was (mean±1.95SD) 0.4±2.7 mmHg. The probability of obtaining this level of agreement between PETCO₂ and PaCO₂ by chance is <0.0001.

Observations: Rebreathing at end-inspiration reduces PET-aCO₂ to a clinically useful range in a ventilated animal model with lung pathology and cardiac shunting.

Animal Preparation: Anesthesia was induced with a 0.2 ml/kg mixture of ketamine 58.8 mg/ml, acepromazine 1.18 mg/ml, and atropine 90 μg/ml administered by intramuscular injection, followed by 3% isoflurane in O₂ to deepen anesthesia for surgical preparation. A catheter was inserted into the ear vein for continuous intravenous infusion anesthesia (22 mg/kg/h ketamine and 1 mg/kg/h midazolam). A 4 mm i.d. uncuffed pediatric endotracheal tube and a catheter for gas and pressure sampling were placed in the trachea via a tracheotomy. A catheter for arterial blood sampling was inserted into the carotid artery via surgical cut-down.

Study: Piglets were initially mechanically ventilated with an O₂ and air mixture in pressure control mode with peak inspiratory pressures between 15-20 cmH₂O, PEEP 0 cmH₂O, frequency of 25-30/min, and inspiration:expiration ratio of 1:3. A secondary circuit providing gas from a gas blender, followed by previous exhaled gas (“sequential rebreathing”) (FIG. 5) was interposed between the ventilator and the endotracheal tube. Peak inspiratory pressures were adjusted to induce rebreathing as evidenced by a rise in the capnograph tracing during the latter part of inspiration. Tidal volumes required to achieve rebreathing, ranged between 80-150 mL. The intra-tracheal catheter was used to monitor airway pressures and sample tidal gas for partial pressure analysis. After the piglets were stabilized on the ventilator, pancuronium bromide 0.2 mg/kg was administered intravenously as a bolus followed by an infusion at 1 mg/kg/h for the duration of the experiment.

Terminal Rebreathing while Targeting End-Tidal Gas Concentrations

In FIG. 5 we present an example of a simple improvised mechanism that can be applied to most ventilatory circuits (including anesthesia circuits) and adjusted to induce rebreathing at the end of the breath, for controlled ventilation without patient triggering. However, our aim was to study PET-aCO₂ at a wide range of PETCO₂ and PETO₂ rather than at the one level. The method of Slessarev et al.⁷ used to target end-tidal values already incorporates rebreathing before termination of each inspiration as part of its targeting strategy.

### Study Protocol

VA was varied systematically in the following three experiments to test the effect of delivering rebreathed gas at the end of inspiration on PET-aCO₂ (FIG. 2):
1. Isoxic ΔPCO₂: From a VA producing a baseline condition (PETCO₂=40 mmHg, PETO₂ 100 mmHg), VA was systematically altered to target isoxic step increases and decreases of 10 mmHg PETCO₂ in random order, returning to baseline after each step change.
2. Isocapnic ΔPO₂. From baseline, VA was changed systematically to target isocapnic step increases in PETO₂ to 500 mmHg (protocol 2a) and step decreases in PETO₂ to 35 mmHg (protocol 2b).
3. ΔPCO₂+ΔPO₂. From baseline, VA was changed to target PETCO₂ 50 mmHg+PETO₂ 300 mmHg, and PETCO₂ 30 mmHg+PETO₂ 60 mmHg in a block fashion, returning to baseline between steps.

Changes in target PETCO₂ required adjustments of tidal volume and frequency settings of the ventilator to assure an element of rebreathing (as evidenced by an increase of the inspired PCO₂ on the capnograph) with every breath. Every step change was maintained for 3 min, and PETCO₂ was taken as the average PETCO₂ of all breaths during the last minute of every step. An arterial blood sample was drawn during the last minute of each step and analyzed within 30 min of collection (ABL 700, Radiometer Copenhagen, Denmark).

### Statistics

Statistical analysis of the data was performed using the SAS System v.9.1.3 (SAS Institute Inc, Cary N.C., USA). A series of mixed-effect repeated measures models (MMRMs) was performed to determine whether differences in PETCO₂ and PaCO₂ values were significantly greater than zero, and whether the magnitude of these differences varied across sequences, and across target PCO₂ and PO₂ levels. A subject identifier was included as a random effect in each of these models to account for the relatedness of observations taken on the same subject.

Two separate model analyses were conducted, the first to examine PET-aCO₂ as a function of sequence, and the second to examine PET-aCO₂ as a function of target PETCO₂. Bonferroni-adjusted pairwise comparisons were used to examine whether PET-aCO₂ was significantly smaller in the sequence in which both PCO₂ and PO₂ were varied than in the sequence when PETO₂ was maintained constant. A Bland-Altman analysis⁸ was used to calculate the limits of agreement between PETCO₂ and PaCO₂. Data are presented as means±SD.

### Results

Table 1 lists the differences between measured PETCO₂ and PaCO₂ for all three protocols:

### Agreement Between PETCO₂ and PaCO₂

In every instance, PETCO₂ moved in the same direction as PaCO₂. The current example elaborating on an animal model is analogous to a clinical study in which each animal represents a single patient studied over time and at various levels of ventilation. The animals had various combinations and severity of underlying pulmonary disease and cardiac shunts. The animals may also have had undetermined changes in cardio-pulmonary pathophysiology as a result of the severe hypoxia, hypercarbia and hypocarbia induced in Protocol 3. Nevertheless, Bland-Altman analysis of our data indicated that the agreement between PETCO₂ and PaCO₂ was 0.4±2.7 mmHg (FIG. 5). The probability of obtaining our level of agreement between PETCO₂ and PaCO₂ by chance is <0.0001.⁹

The consistently small Pet-aCO₂ in our study contrasts with those of most other studies in which PET-aCO₂ varies widely between subjects and in the same subjects over time. McDonald et al.² studied 1708 sample pairs of PETCO₂ and PaCO₂ in 129 children in an intensive care unit; PET-aCO₂ ranged between 0 to >−30 mmHg and only 74% of samples changed in the same direction. Tobias et al.³ reported a range of PET-aCO₂ of 5 to −22 mmHg in 100 sample sets in 25 infants and toddlers. For perspective, the studies by McDonald et al.² and Tobias et al.³ suggested that even the broad Pet-aCO₂ in their studies of −4.7±8.2 mmHg and −6.8±5.1 mmHg respectively were still within a “clinically acceptable” range. Yamanaka et al.¹⁰, in a study of 17 ventilated adults in a critical care unit, found that the correlation between PETCO₂ and PaCO₂ was too poor for PETCO₂ to be used even as an indicator of direction of changes of PaCO₂. Others too have found poor, or no, correlations between PETCO₂ and PaCO₂ in adults with multi-system disease¹¹, trauma⁴, undergoing neurosurgeryl^{12;13}, as well as in dogs with healthy lungs¹⁴ or lungs with oleic acid-induced ARDS¹⁵. That our findings differ from those in the literature is likely due to the simple expedient of administering previously exhaled gas at the end of each inspiration, thereby reducing mean±1.95SD PET-aCO₂ to 0.4±2.7 mmHg despite considerable pulmonary and circulatory pathology.

### REFERENCE LIST

Berkenbosch J W, Lam J, Burd R S, Tobias J D: Noninvasive monitoring of carbon dioxide during mechanical ventilation in older children: end-tidal versus transcutaneous techniques. Anesth. Analg. 2001; 92: 1427-31
2. McDonald M J, Montgomery V L, Cerrito P B, Parrish C J, Boland K A, Sullivan J E: Comparison of end-tidal CO2 and Paco2 in children receiving mechanical ventilation. Pediatr. Crit. Care Med. 2002; 3: 244-9
3. Tobias J D, Meyer D J: Noninvasive monitoring of carbon dioxide during respiratory failure in toddlers and infants: end-tidal versus transcutaneous carbon dioxide. Anesth. Analg. 1997; 85: 55-8
4. Russell G B, Graybeal J M: Reliability of the arterial to end-tidal carbon dioxide gradient in mechanically ventilated patients with multisystem trauma. J. Trauma 1994; 36: 317-22
5. Bowie J R, Knox P, Downs J B, Smith R A: Rebreathing improves accuracy of ventilatory monitoring 1. J. Clin. Monit. 1995; 11: 354-7
6. Ito S, Mardimae A, Han J, Duffin J, Wells G, Fedorko L, Minkovich L, Katznelson R, Meineri M, Arenovich T, Kessler C, Fisher J A: Non-invasive prospective targeting of arterial P(CO2) in subjects at rest. J. Physiol 2008; 586: 3675-82
7. Slessarev M, Han J, Mardimae A, Prisman E, Preiss D, Volgyesi G, Ansel C, Duffin J, Fisher J A: Prospective targeting and control of end-tidal CO2 and O2 concentrations. J Physiol 2007; 581: 1207-19
8. Bland J M, Altman D G: Statistical methods for assessing agreement between two methods of clinical measurement. Lancet 1986; 1: 307-10
9. Preiss D, Fisher J: A measure of confidence in Bland-Altman analysis for the interchangeability of two methods of measurement. J. Clin. Monit. Comput. 2008; 22: 257-9
10. Yamanaka M K, Sue D Y: Comparison of arterial-end-tidal PCO2 difference and dead space/tidal volume ratio in respiratory failure. Chest 1987; 92: 832-5
11. Hoffman R A, Krieger B P, Kramer M R, Segel S, Bizousky F, Gazeroglu H, Sackner M A: End-tidal carbon dioxide in critically ill patients during changes in mechanical ventilation. Am Rev. Respir. Dis. 1989; 140: 1265-8
12. Russell G B, Graybeal J M: The arterial to end-tidal carbon dioxide difference in neurosurgical patients during craniotomy. Anesth. Analg. 1995; 81: 806-10
13. Russell G B, Graybeal J M: End-tidal carbon dioxide as an indicator of arterial carbon dioxide in neurointensive care patients. J Neurosurg Anesthesiol. 1992; 4: 245-9
14. Neto F J, Carregaro A B, Mannarino R, Cruz M L, Luna S P: Comparison of sidestream capnograph and a mainstream capnograph in mechanically ventilated dogs. J Am Vet Med Assoc 2002; 221: 1582-5
15. Murray I P, Modell J H, Gallagher T J, Banner M J: Titration of PEEP by the arterial minus end-tidal carbon dioxide gradient. Chest 1984; 85: 100-4
16. Bynum L J, Wilson J E, Ill, Pierce A K: Comparison of spontaneous and positive-pressure breathing in supine normal subjects. J. Appl. Physiol 1976; 41: 341-7
17. Moens Y, Lagerweij E, Gootjes P, Poortman J: Influence of tidal volume and positive end-expiratory pressure on inspiratory gas distribution and gas exchange during mechanical ventilation in horses positioned in lateral recumbency. Am J Vet Res. 1998; 59: 307-12
18. Lindahl S G: Oxygen consumption and carbon dioxide elimination in infants and children during anaesthesia and surgery. Br. J Anaesth. 1989; 62: 70-6
19. Fletcher R: The relationship between the arterial to end-tidal PCO2 difference and hemoglobin saturation in patients with congenital heart disease. Anesthesiology 1991; 75: 210-6
20. Read D J: A clinical method for assessing the ventilatory response to carbon dioxide. Australas. Ann. Med. 1967; 16: 20-32
21. Casey K, Duffin J, McAvoy G V: The effect of exercise on the central-chemoreceptor threshold in man. Journal of Physiology 1987; 383: 9-18
22. Mohan R M, Amara C E, Cunningham D A, Duffin J: Measuring central-chemoreflex sensitivity in man: rebreathing and steady-state methods compared. Respir. Physiol 1999; 115: 23-33
23. Banzett, R. B., Garcia, R. T., and Moosavi, S. H. Simple contrivance “clamps” end-tidal PCO2 and PO2 despite rapid changes in ventilation. Journal of Applied Physiology 88, 1597-1600. 2000. Ref Type: Journal (Full)
24. Sommer L Z, Iscoe S, Robicsek A, Kruger J, Silverman J, Rucker J, Dickstein J, Volgyesi G A, Fisher J A: A simple breathing circuit minimizing changes in alveolar ventilation during hyperpnoea. Eur. Respir. J. 1998; 12: 698-701
25. Somogyi R B, Vesely A E, Preiss D, Prisman E, Volgyesi G, Azami T, Iscoe S, Fisher J A, Sasano H: Precise control of end-tidal carbon dioxide levels using sequential rebreathing circuits. Anaesth. Intensive Care 2005; 33: 726-32
26. meida-Junior A A, da Silva M T, Almeida C C, Ribeiro J D: Relationship between physiologic deadspace/tidal volume ratio and gas exchange in infants with acute bronchiolitis on invasive mechanical ventilation. Pediatr. Crit. Care Med 2007; 8: 372-7
27. Hillier S C, Badgwell J M, McLeod M E, Creighton R E, Lerman J: Accuracy of end-tidal PCO2 measurements using a sidestream capnometer in infants and children ventilated with the Sechrist infant ventilator. Can. J. Anaesth. 1990; 37: 318-21
28. Burrows F A: Physiologic dead space, venous admixture, and the arterial to end-tidal carbon dioxide difference in infants and children undergoing cardiac surgery. Anesthesiology 1989; 70: 219-25
29. Hedenstierna G, McCarthy G: The effect of anaesthesia and intermittent positive pressure ventilation with different frequencies on the anatomical and alveolar deadspace. Br. J Anaesth. 1975; 47: 847-52
30. Short J A, Paris S T, Booker P D, Fletcher R: Arterial to end-tidal carbon dioxide tension difference in children with congenital heart disease. Br. J. Anaesth. 2001; 86: 349-53
31. Moss A J, EMMANOUILIDES G, DUFFIE E R, Jr.: Closure of the ductus arteriosus in the newborn infant. Pediatrics 1963; 32: 25-30
32. EMMANOUILIDES G C, Moss A J, DUFFIE E R, Jr., DAMS F H: PULMONARY ARTERIAL PRESSURE CHANGES IN HUMAN NEWBORN INFANTS FROM BIRTH TO 3 DAYS OF AGE. J Pediatr. 1964; 65: 327-33
33. ito S, Mardimae A, Han J S, Duffin J, Wells G, Fedorko L, Minkovich L, Katznelson R, Meineri M, Kessler K, Fisher J A: Non-invasive prospective targeting of arterial PCO2 in subjects at rest. Journal of Physiology 2008; 586: 3675-82
34. Badgwell J M, McLeod M E, Lerman J, Creighton R E: End-tidal PCO2 measurements sampled at the distal and proximal ends of the endotracheal tube in infants and children. Anesth. Analg. 1987; 66: 959-64
35. Badgwell J M, Heavner J E, May W S, Goldthorn J F, Lerman J: End-tidal PCO2 monitoring in infants and children ventilated with either a partial rebreathing or a non-rebreathing circuit. Anesthesiology 1987; 66: 405-10
36. Winter J D, Fierstra J, Dorner S, Fisher J A, St Lawrence K S, Kassner A: Feasibility and precision of cerebral blood flow and cerebrovascular reactivity MRI measurements using a computer-controlled gas delivery system in an anesthetised juvenile animal model. J Magn Reson. Imaging 2010; 32: 1068-75

We claim:

1. The use of a gas delivery system optionally comprising a ventilator to determine arterial blood gas concentrations in a ventilated patient, the gas delivery system organized to deliver to the patient for a series, optionally a plurality of consecutive inspiratory cycles, one or more gases comprising carbon dioxide to diminish or minimize the partial pressure gradient between the patient's PETCO₂ and PaCO₂.

2. The use according to claim 1, wherein the gas delivery system is organized to sequentially deliver for the first portion of each of the respective inspiratory cycles a first gas having a first gas composition and for the second portion of each of the respective inspiratory cycles a second gas which has partial pressure of carbon dioxide which is relatively higher than that of the first gas, optionally the delivery of carbon dioxide for a plurality of inspiratory cycles simulates rebreathing for a portion of each inspiratory cycle or is accomplished by delivering as the second gas a gas having a PCO₂ approximating the patient's PETCO₂ in the expiratory cycle immediately preceding the respective inspiratory cycle. The optimal value may be equal to or approximate the PaCO₂.

3. The use according to claim 1, wherein the gas delivery system comprises a gas injector for injecting a gas comprising CO₂ into an inspiratory gas delivered by the ventilator.

4. The use according to claim 1, wherein the gas delivery system includes a breathing circuit comprising an inspiratory limb for establishing a fluidic connection between the ventilator and a patient airway interface and an expiratory limb establishing a fluidic connection between the ventilator and the patient airway interface, and wherein breathing circuit is organized to redirect ventilator flow from the inspiratory limb to the expiratory limb during inspiration to drive exhaled gas in the expiratory limb towards the patient airway interface as part of each consecutive inspiratory cycle.

5. The use according to claim 4, wherein the ventilator flow is redirected from the inspiratory limb to the expiratory limb in response to airway pressure.

6. The use according to claim 4, wherein the breathing circuit comprises a valve for channeling airflow from the ventilator to one of the limbs for the first portion of each inspiratory cycle and for reversibly diverting airflow generated by the ventilator to the other limb during the second portion of each inspiratory cycle. Optionally, the breathing circuit is connected to a ventilator and organized to sequentially deliver a first gas and then a CO₂ containing gas down the expiratory limb.

7. The use according to claim 6, wherein the expiratory limb includes a expiratory gas reservoir portion and wherein the valve is interposed between the ventilator and the expiratory gas reservoir portion for driving expiratory gas contained in the expiratory gas reservoir portion towards the patient airway interface during the second portion of each of the inspiratory cycles.

8. The use according to claim 7, wherein the valve comprises a first airway fluidically connectable between the ventilator and the inspiratory limb and a second airway fluidically connectable between the ventilator and the expiratory limb and at least one air flow blocking member.

9. The use according to claim 8, wherein the at least one airflow blocking member is movable between a first airway occluding position and a second airway occluding position.

10. The use according to claim 9, wherein the valve comprises at least one biasing element for biasing the airflow blocking member towards the second airway occluding position, optionally during the first portion of each inspiratory cycle.

11. The use according to claim 10, wherein the valve comprises at least one air pressure responsive member operatively connected to the at least one airflow blocking member and movable therewith between the first airway occluding position and the second airway occluding position.

12. The use according to claim 10, wherein the airflow blocking member is driven towards the first airway occluding position in response to an increase in airway pressure in the inspiratory limb.

13. A method for determining arterial blood gas concentrations in a ventilated patient with pulmonary dysfunction preliminary to a diagnostic assessment of the patient's respiratory condition, comprising the step of delivering to the subject for a plurality of inspiratory cycles one more gases comprising carbon dioxide in an amount sufficient to equilibrate the patient's PETCO₂ and arterial PCO₂ such that the patient's PETCO₂ is a clinically reliable approximation of the patient's PaCO₂.

14. A method according to claim 13, further comprising the step of measuring the patient's PETCO₂ after the plurality of inspiratory cycles.

15. A valve for use in conjunction with a ventilator breathing circuit of the type having an inspiratory limb segment for establishing a fluidic connection between the ventilator and a patient airway interface and an expiratory limb segment establishing a fluidic connection between the ventilator and the patient airway interface, the valve adapted to redirect ventilator flow from the inspiratory limb to the expiratory gas reservoir portion during inspiration to drive exhaled gas in the expiratory limb towards the patient airway interface during an inspiratory cycle.

16. A valve according to claim 15, comprising a first airway fluidically connectable between the ventilator and the inspiratory limb and a second airway fluidically connectable between the ventilator and the expiratory limb, at least one air flow blocking member movable between a first airway occluding position and a second airway occluding position.

17. A valve according to claim 16, comprising at least one biasing element for biasing the airflow blocking member towards the second airway occluding position.

18. A valve according to claim 17, comprising at least one air pressure responsive member operatively connected to the at least one airflow blocking member and movable therewith between the first airway occluding position and the second airway occluding position.

19. A valve according to claim 18, wherein the airflow blocking member is adapted to be driven towards the first airway occluding position in response to an increase in airway pressure in the inspiratory limb.

20. A breathing circuit for use in conjunction with a ventilator comprising:
An expiratory limb for establishing a fluidic connection between the ventilator and a patient airway interface;
An inspiratory limb for establishing a fluidic connection between the ventilator and a patient airway interface;
A valve for channeling airflow from the ventilator to one of the limbs for a first portion of an inspiratory cycle and for reversibly diverting airflow generated by the ventilator to the other of the limb during any second portion of an inspiratory cycle.

21. A breathing circuit according to claim 20, wherein the expiratory limb includes a expiratory gas reservoir portion proximal to the patient airway interface and wherein the valve is interposed between the ventilator and the expiratory gas reservoir portion of the expiratory limb for driving expiratory gas contained in the expiratory gas reservoir portion towards the patient airway interface during one of the first or second portions an inspiratory cycle.

22. The use of a gas delivery system to determine an arterial blood gas concentration in a patient with pulmonary dysfunction, the gas delivery system organized to deliver to the patient for one or a series, optionally a plurality of consecutive inspiratory cycles, one more gases comprising carbon dioxide to diminish or minimize the partial pressure gradient between the patient's PETCO₂ and arterial PCO₂.

23. The use according to claim 22, further comprising the step of ascertaining the value of PETCO₂ at the end a plurality of inspiratory cycles.

24. The use according to claim 22, wherein the gas delivery system is organized to deliver a first gas for at least a portion of each inspiratory cycle and the patient's exhaled gas, optionally for each inspiratory cycle, the gas exhaled at the end the immediately preceding inspiratory cycle, for at least a portion, optionally a different portion, of each inspiratory cycle.

25. The use according to claim 22, wherein a value of PETCO₂ is obtained at the end of one or more of a plurality and inspiratory cycles and optionally wherein said value is later used to make a diagnostic evaluation of the patient's condition.

26. The use according to claim 1, in a patient with pulmonary disease, or a systemic disease having symptoms or the treatment of which affects the distribution of blood flow in the lung or distribution of ventilation or both.

27. The use according to claim 26, wherein the gas delivery system is organized to deliver exhaled gas to the patient for a series, optionally a plurality of consecutive inspiratory cycles, to diminish or minimize the partial pressure gradient between the patient's PETCO₂ and PaCO₂.

28. The use according to claim 27, wherein the breathing circuit is designed to comingle an inspiratory gas with a suitable amount of exhaled gas.

29. The use according to claim 28, wherein exhaled gas is diverted into an inspiratory limb of a breathing circuit.

30. A breathing circuit for use in conjunction with a ventilator comprising:
Means (optionally a conduit) defining an expiratory gas airflow pathway;
Means (optionally a conduit) defining an inspiratory gas airflow pathway;
Means for mingling (optionally channeling via a conduit and/or valve) inspiratory gas and expiratory gas in an amount sufficient to equilibrate the patient's PETCO₂ and arterial PCO₂ such that the patient's PETCO₂ is a clinically reliable approximation of the patient's PaCO₂, optionally by channeling expiratory gas into the inspiratory gas, optionally in the inspiratory gas flow pathway.
